SEQUENCE LISTING (1 sequence)

SEQ ID NO: 1 - protein, 210 aa, Homo sapiens
MPPYTVVYFPVRGRCAALRMLLADQGQSWKEEVVTVETWQEGSLKASCLYGQLPKFQDGDLTLYQSNTILRHLGRTLGLYGKDQQEAALVDMVNDGVEDLRCKYISLIYTNYEAGKDDYVKALPGQLKPFETLLSQNQGGKTFIVGDQISFADYNLLDLLLIHEVLAPGCLDAFPLLSAYVGRLSARPKLKAFLASPEYVNLPINGNGKQ

(19) United States
(12) Patent Application Publication
(10) Pub. No.: US 2023/0152312 A1
(43) Pub. Date: May 18, 2023

(54) DEVICES AND METHODS FOR TREATING ISCHAEMIA AND ACUTE RESPIRATORY DISTRESS SYNDROMES

(71) Applicant: Galaxy CCRO, Inc., Corpus Christi, TX (US)

(72) Inventors: Ian Hugo PIKE, Tonbridge Kent (GB); Terry ELDER, Corpus Christi, TX (US); Maxime LAROCHE, Guipry (FR); Milovan STANKOV, Guipry (FR)

(21) Appl. No.: 17/911,959

(22) PCT Filed: Feb. 12, 2021
(86) PCT No.: PCT/US2021/018015

§ 371 Date: Sep. 15, 2022

Related U.S. Application Data
(60) Provisional application No. 62/977,133, filed on Feb. 14, 2020.
(60) Provisional application No. 63/014,088, filed on Apr. 22, 2020.

(51) Int. Cl. G01N 33/543

(52) U.S. Cl. CPC G01N 33/54388; G01N 2333/91177; G01N 2800/125; G01N 2800/2871; G01N 2800/52

(57) ABSTRACT

Devices and methods for conducting lateral flow immunochromatographic assays may be used, in some aspects, to measure the level of glutathione-S-transferase pi in a biological fluid collected from a human subject suspected of having had a stroke in order to determine whether the subject has had a stroke or to aid in the selection and administration of a treatment for the suspected stroke. In other aspects, such devices may be used to diagnose, monitor, and/or treat acute respiratory distress syndromes (ARDS), such as the novel coronavirus designated COVID-19.

### CROSS-REFERENCE TO RELATED APPLICATIONS

This application is a National Stage Entry of PCT/US2021/018015, filed Feb. 12, 2021, which claims the benefit of U.S. Provisional Application No. 62/977,133, filed on Feb. 14, 2020, and 63/014,088, filed on Apr. 22, 2020, the contents of each of which are hereby incorporated by reference in their entirety.

### TECHNICAL FIELD

The present disclosure relates to lateral flow assays, particularly, immunoassays. More specifically, the disclosure provides devices and methods for determining the concentration or level of glutathione S-transferases pi (GST-Pi) present in a sample of a biological fluid (e.g., to diagnose, monitor, and/or treat various conditions).

### BACKGROUND

The glutathione S-transferases (GSTs) are a family of enzymes known to be involved in the detoxification and, in some cases, activation of a wide variety of chemical compounds. The GSTs perform this function by catalyzing the conjugation of reduced glutathione with many hydrophobic and electrophilic compounds. Based on their biochemical, immunologic, and structural properties, the soluble GSTs are organized into five main classes: alpha, mu, pi, sigma, and theta. The human glutathione S-transferase pi gene (GSTP1) is a polymorphic gene that encodes a 210 amino acid protein (NCBI RefSeq No. NP_000843.1; SEQ ID NO: 1), as well as variant alleles that are thought to have implications for xenobiotic metabolism, susceptibility to cancer, and various other diseases.

At least one study has identified GST-Pi as a prospective biomarker for detecting whether a human subject has suffered from a stroke. In particular, a paper by Turck et al., entitled “Blood glutathione S-transferase-7c as a time indicator of stroke onset,” PloS One 7.9 (2012), reported that GST-Pi can be used as a biomarker to determine the onset of a stroke. Stroke remains a global healthcare challenge and is one of the leading causes of death and serious long-term disability in the United States. Strokes can be classified into two main categories, ischemic strokes caused by blockage of an artery (or, in some instances, a vein) and hemorrhagic strokes caused by bleeding. An ischemic stroke may result from a barrier within a blood vessel supplying blood to the brain (thrombotic stroke) or as a result of a blood vessel in the brain being blocked by an embolus produced from a clot somewhere else in the body which has traveled to the brain (embolic stroke). These blockages deprive the brain of necessary oxygen and may result in permanent brain cell death in and around the affected areas. In recent years, thrombolytic therapy (i.e., the administration of one or more thrombolytic agents to break up or dissolve blood clots) has emerged as a potential breakthrough treatment for stroke. However, current studies suggest that the effectiveness of thrombolytic therapy rapidly decreases as time progresses after the initial onset of a stroke.

Currently, stroke onset may be determined using magnetic resonance imaging (“MRI”) techniques. However, these methods are non-ideal in view of the fact that access to MRI equipment is limited. Many hospitals and other treatment facilities do not have MRI equipment available. Moreover, this equipment is too large, complex, and expensive to be practical for home installation or field use (e.g., by paramedics). MRI-based methods are also known to display low sensitivity as a diagnostic for stroke onset due to image quality issues. In recent years, researchers have identified a variety of biomarkers that may have use as a clinical diagnostic for determining stroke onset. For example, GST-Pi was identified by Turck et al. as one of many potential biomarkers, as noted above. However, to date preliminary studies by researchers in this area have yet to provide viable point-of-care (POC) devices for the diagnosis and treatment of strokes and other similar medical events based on GST-Pi.

GST-Pi has also been implicated in the process of platelet activation, which is a key step required in the formation of a thrombus (blood clot). In particular, activation of platelets during the clotting process is associated with the release of GST-Pi.

Thrombocytopenia (a reduction in platelet levels) is known to be a common feature of several disorders including bacterial and viral sepsis and acute respiratory distress syndromes (ARDS) caused by novel coronaviruses such as those associated with the severe acute respiratory syndrome (SARS) epidemic that occurred during the early 2000's, as well as the COVID-19 pandemic of 2020. By way of example, the reduction in platelet levels seen in COVID-19 infection is believed to be associated with endothelial damage caused by viral infection and exacerbated by intubation. This in turn leads to activation of platelets, followed by aggregation and thrombosis in the lung. In other studies, more widespread evidence of platelet dysregulation has been reported. Disseminated intravascular coagulation (DIC) has been described in over 70% of COVID-19 patients who die, compared with less than 1% of survivors, and is being seen predominantly as a pro-thrombotic event with high levels of venous thromboembolism, elevated D-Dimer and fibrinogen levels and intravascular thrombosis. These findings have led to evaluation of fibrinolytic and thrombotic therapies in acute respiratory distress with some evidence of success.

The administration of thromboprophylaxis treatment with Nadoparin (heparin) to COVID-19 patients has been found not to prevent thrombotic complications in a recent Dutch study where 31% of ICU patients had thrombosis. Alternative therapeutic strategies to break up clots in COVID-19 patients using tissue plasminogen activator (rTPA) or to prevent platelet activation using aspirin and/or Plavix are ongoing at this time.

There is also growing evidence of a link between inflammation and platelet activation regulated by the JAK-STAT signaling pathway. STAT-3, a member of this pathway has been shown to cause enhanced platelet activation by collagen and is linked with increased levels of clotting in inflammatory conditions (Zhou et al. 2013). Inhibition of JAK2-STAT3 has also been shown to reduce platelet activation in vitro (Yuan et al. 2015) and JAK2 inhibitors such as barcitinib are showing promise in the treatment of COVID-19. Whilst levels of JAK2 and STAT3 are difficult to measure in peripheral samples such as blood and broncheoalveolarfluids, the high levels of GST-Pi released by activated platelets are readily detectable.

There remains therefore, a need to provide a means of assessing COVID-19 patients to determine their coagulation status and stratify their risk of significant worsening if disease is due to uncontrolled clotting. In this context, administration of anticoagulants and thrombolytics are expected to deliver improved outcomes. The GST-Pi lateral flow device provided herein provides such as a stratification tool.

### BRIEF SUMMARY OF EXEMPLARY ASPECTS

In view of the shortcomings of the prior art, the present disclosure provides devices and methods that may be used to diagnose, monitor, and/or treat cerebrovascular accidents, such as stroke and sub-arachnoid hemorrhage, ARDS, such as the novel coronavirus designated COVID-19, and sepsis caused by bacterial or viral infection, all conditions associated with inflammation, platelet activation and abnormal clotting.

In particular, there exists a need for portable, low-cost, and sensitive POC devices that can be used for determining whether and when a human subject has had a stroke, or in other aspects, for determining the current status, likelihood of progression to severe disease, and monitoring of treatment effects, in a subject suffering from an ARDS. The present disclosure addresses both of these needs, in addition to providing other benefits, by disclosing portable lateral flow devices and methods which can be used to measure the concentration of GST-Pi in a biological fluid collected from a subject. Such devices are advantageous in that they can be manufactured at low cost, are portable (e.g., available for use at home or in the field, rather than limited to a hospital setting), and can be used to rapidly measure GST-Pi concentrations with a high sensitivity (e.g., in some aspects, the disclosed devices are capable of measuring GST-Pi concentrations above approximately 20 ng/ml).

The portable lateral flow devices described herein are particularly advantageous for the diagnosis, monitoring, and treatment of thromboembolic complications associated with ARDS, given that that they can be used directly at the POC and only require a small sample of blood that can be drawn by a finger prick or from an indwelling catheter, if available. Such assays require no additional equipment and results can be read within 5 to 15 minutes with a positive staining in the GST-Pi line confirming thromboembolic complications. Additional benefits will become apparent in view of the following description and the accompanying drawings.

In a first general aspect, the disclosure provides a lateral flow immunoassay device for detecting and/or measuring the concentration of GST-Pi in a sample of a biological fluid, the device comprising a test strip for detecting GST-Pi in the sample, wherein the test strip comprises: a) a sample pad, wherein the sample pad comprises an absorbent material and is configured to receive the sample; b) a conjugate pad configured to store a detection antibody specific for GST-Pi and to release at least a portion of the stored detection antibody in the presence of a liquid, wherein the detection antibody is conjugated to a colored label moiety; and c) a colorimetric indicator site positioned downstream from the absorbent pad, wherein the colorimetric indicator site comprises a capture antibody specific for GST-Pi fixed to the test strip.

In some aspects, the device further comprises a lancet with a capillary channel. In some aspects, at least a portion of the test strip comprises a nitrocellulose membrane (e.g., with an average pore size of 5, 10, 15, 20, 25, or 30 μm). In some aspects, the capture antibody and the detection antibody are configured to bind to different moieties of GST-Pi. The capture and detection antibodies may be independently selected from any of the antibodies described herein. In some aspects, the colored label moiety comprises a gold or latex nanoparticle, optionally present at an optical density of 1, 2, 3, 4, 5, 6, 7, 8, 9, or 10. In some aspects, the capture antibody and/or the detection antibody are present at a concentration of 0.1, 0.2, 0.3, 0.4, 0.5, 0.6, 0.7, 0.8, 0.9, 1.0, 1.1, 1.2, 1.3, 1.4 or 1.5, 1.6, 1.7, 1.8, 1.9 or 2.0 mg/mL. In some aspects, the biological fluid comprises a sample of whole blood from a human subject.

In another general aspect, the disclosure provides a method for determining whether a subject has had a stroke or ischemic attack, comprising: a) obtaining a sample of a biological fluid from a subject suspected of having had a stroke or ischemic attack; b) applying at least a portion of the sample to a lateral flow device described herein; and c) detecting a level and/or concentration of GST-Pi in the sample using the lateral flow device. In some aspects, such methods may further comprise: d) determining an estimated time of onset of the stroke or ischemic attack based on the level and/or concentration of GST-Pi detected in step c). In still further aspects, such methods may comprise: d) determining an estimated time of onset of the stroke or ischemic attack based on the level and/or concentration of GST-Pi detected in step c); and e) selecting a treatment for the subject based on the estimated time of onset determined in step d). A treatment for the subject may comprise administration of a thrombolytic therapy.

In another general aspect, the disclosure provides a method for determining the current status and/or likelihood of progression to severe disease, of a human subject suffering from an ARDS, comprising: a) obtaining a sample of a biological fluid from a subject suspected of having disseminated vascular coagulation; b) detecting or measuring the level of GST-Pi in the sample obtained in step a); and c) determining the current status and/or likelihood of progression to severe disease, of the human subject, based on the level of GST-Pi detected or measured in step b). Such methods may also be used to monitor the effect of treating a human subject for an ARDS, e.g., by measuring the level of GST-Pi over time, before and/or after a treatment, etc., as described herein.

In some aspects, the disclosure also provides methods of treating a subject suffering from an ARDS, comprising: a) obtaining a sample of a biological fluid from a subject suspected of having disseminated vascular coagulation; b) detecting or measuring the level of GST-Pi in the sample obtained in step a); and selecting a JAK-STAT inhibitor, thrombolytic or anti-platelet treatment for the subject based on the detection and/or measurement of elevated GST-Pi levels in step b).

This simplified summary of exemplary aspects of the disclosure serves to provide a basic understanding of the invention. This summary is not an extensive overview of all contemplated aspects, and is intended to neither identify key or critical elements of all aspects nor delineate the scope of any or all aspects of the invention. Its sole purpose is to present one or more aspects in a simplified form as a prelude to the more detailed description of the invention that follows. To the accomplishment of the foregoing, the one or more aspects of the invention include the features described and particularly pointed out in the claims.

### BRIEF DESCRIPTION OF THE DRAWINGS

FIG. 1 is a diagram showing the structure of the test strip portion of an exemplary lateral flow device according to the disclosure.

FIG. 2 is a chart summarizing the results of comparative tests using pairs of the various capture and detection antibodies described herein.

FIG. 3 is a chart showing the results of an assay which evaluated the effectiveness of using a detection antibody conjugated to nanoparticles present at different optical density levels.

FIG. 4 is a graph showing the results of a study that evaluated the use of exemplary lateral flow devices according to the present disclosure to measure the concentration of GST-Pi in whole human blood samples spiked with known quantities of GST-Pi.

FIG. 5 is a flowchart showing an exemplary protocol for preparing blood fractions to evaluate GST-Pi release from platelets in a biological sample obtained from a human subject.

FIG. 6 is a graph showing that levels of GST-Pi correlate with the platelet levels in blood fractions prepared from a biological sample obtained from a human subject.

FIG. 7 is a photograph showing the results of measuring GST-Pi in plasma samples drawn from patients with clinically-confirmed stroke at early (<3 hour) and late (>6 hour) time-points.

FIG. 8A is a graph showing the results of a study that evaluated the use of exemplary lateral flow devices according to the present disclosure to measure the concentration of GST-Pi in serum samples drawn from patients with SARS-Cov-2 (COVID-19) infection and healthy controls. The median value in the COVID-19 group is elevated compared with healthy controls, and individual patients demonstrated different temporal evolution of the GST-Pi signal.

FIG. 8B is a graph showing the GST-Pi score for each of 64 serum samples from 30 individual COVID-19 infected patients undergoing treatment in hospital. 15 individual samples showed elevated levels (Score ≥6) and these were drawn from 9 separate patients.

### DETAILED DESCRIPTION OF EXEMPLARY ASPECTS

In the United States, approximately 675,000 people suffer an ischemic stroke each year. Approximately 20% of stroke cases lack an established time of onset. For example, “wake-up stroke” (WUS), defined as the situation where a patient awakens with stroke symptoms that were not present prior to falling asleep, represents roughly 20% of acute ischemic strokes. Subjects who have suffered a WUS are ineligible for thrombolysis because of the risk of bleeding, if treatment has been delayed too long. Moreover, delays in transferring patients to specialized stroke centers also result in many patients being outside of the window for thrombolytic treatment. As such, only 15% of ischemic stroke patients receive this life-changing treatment. Prompt administration of thrombolytic therapy is critical because every minute of brain hypoxia kills 2 million brain cells, and treatment to dissolve clots administered within 2 hours of stroke onset results in significantly better clinical outcomes.

Rapid POC diagnostic platforms have revolutionized treatment for many pathologies, such as cardiac troponin for myocardial infarction and D-dimer for pulmonary embolism. However, the promise of blood biomarkers to provide early diagnosis and to establish the time of onset of WUS has yet to be realized by prior methods. The present disclosure addresses this issue by providing POC devices that can be used to rapidly and accurately determine stroke onset time, allowing stroke patients to receive a timely diagnosis and correct treatment.

The POC devices described herein use GST-Pi concentration as a biomarker for ischemic or hemorrhagic stroke and may be used to repeatedly track rising levels of GST-Pi in order to determine the time of stroke onset. The concentration of GST-Pi in the blood increases within minutes following an ischemic or hemorrhagic stroke, resolving to baseline levels again by 6 hours. The present disclosure provides lateral flow devices that can be used, e.g., to determine a kinetic profile of GST-Pi levels over the first 60 minutes post-event. These devices may be used in order to determine that onset occurred within less than 3 hours, confirming that a patient is eligible for thrombolytic therapy. The portable and rapid devices described herein are particularly advantageous for field use, but may also be used to increase the speed and accuracy of stroke diagnoses by emergency rooms and trauma centers. It is also particularly advantageous that the devices disclosed herein may be used at regular intervals of 10, 15 or 20 minutes to establish the rate of increase or decrease in levels of GST-Pi in the blood of an individual suspected of having had a stroke or cerebrovascular accident and using this kinetic value to determine the relative time of onset, wherein increasing values represent an active stroke initiated within 3 hours.

It is a further feature of the invention that the device may be used to assess the state of head injury following trauma or collision-induced head injury characterized by concussion. In this respect it is particularly advantageous that results can be obtained within 15 minutes for the assessment of concussion during sports head injury assessment and during emergency settings such as battlefield, road traffic accidents and falls.

As noted above, the present disclosure also provides methods that may be used for determining the current status and/or likelihood of progression to severe disease, of a human subject suffering from an ARDS (e.g., a patient infected with COVID-19). In related aspects, such methods may also be used monitor the status of subjects suffering from an ARDS, or to evaluate the effect of treating such subjects (by measuring the level of GST-Pi at different time-points, before or after the administration of a treatment, etc.). Such methods may be carried out using the portable devices described herein, allowing for assays to be performed directly at the POC, without additional specialized equipment. Moreover, such assays can advantageously be completed within 5 to 15 minutes, allowing for the rapid detection and/or measurement of GST-Pi levels, which can in turn be used to diagnoses or monitor the status of a subject, or to select an appropriate treatment for the subject.

In some aspects, the disclosure provides lateral flow devices comprising a test strip and optionally, an on-board lancet with a capillary channel. The test strip may comprise one or more capillary beds, such as porous paper, or microstructured or sintered polymer(s), which are capable of allowing a liquid to flow laterally by capillary action across at least a portion of the device. Such devices may optionally also be provided in a kit which includes one or more pre-packaged reagents (e.g., a buffer to wet the test strip) and/or an automated flow system. The test strip may include immobilized antibodies to GST-Pi linked to colloidal particles (e.g., gold, latex, or other colored particles) to bind GST-Pi during lateral flow, providing a colorimetric indicator that can be used to detect or measure GST-Pi level in a tested biological fluid. The devices described herein may be used to collect a biological fluid from a subject suspected of having had a stroke (e.g., to collect blood by lancing a finger) or of having an ARDS and/or disseminated vascular coagulation. The collected blood may then be provided to the device along with a buffer or other liquid and allowed to flow across at least a portion of the one or more capillary beds, eventually reaching immobilized antibodies to GST-Pi conjugated to colloidal particles (i.e., resulting in the generation of a colorimetric indicator). In some aspects, one or more of the capillary beds may comprise additional labeled and/or immobilized antibodies (e.g., antibodies to one or more additional components of human blood) in order to provide one or more additional colorimetric indicators, which can serve as a control or other signal.

In some aspects, these lateral flow devices provided herein may generate a colorimetric indicator capable of being detected by visual inspection by a human or an electronic system within 10 minutes or less (e.g., within 1, 2, 3, 4, 5, 6, 7, 8, 9, or 10 minutes). The lateral flow devices described herein may further be configured so that the intensity of this colorimetric indicator varies with GST-Pi concentration in a linear manner, providing a means to assess GST-Pi kinetics over sequential tests (e.g., carried out at different time-points).

While GST-Pi has been recognized as a potential diagnostic biomarker for stroke onset, as well as for platelet activation, prior research has failed to yield any rapid POC assays (e.g., lateral flow devices) capable of leveraging this relationship to aid in the diagnosis and treatment of stroke patients and subjects suffering from an ARDS (e.g., COVID-19). Such devices are now provided, based on the surprising finding that specific pairs of antibodies raised against different portions of GST-Pi, and their orientation in the sandwich format used by the present devices (for capture and detection), can be used to generate a colorimetric indicator capable of detecting GST-Pi concentrations above 20 ng/ml, a cut-off value previously established for ruling out stroke. Indeed, this finding is the product of studies of more than 140 antibody combinations in human plasma spiked with recombinant GST-Pi, the vast majority of which were found to be unsuitable for use in the rapid and sensitive lateral flow assays described herein. In some aspects, the present devices further require specific labels (e.g., gold or red latex) and/or a specific pore size of nitrocellulose used as a capillary bed to maximize signal intensity.

In some aspects, a lateral flow device according to the disclosure may utilize at least one pair of antibodies (for capture and detection of GST-Pi) selected from any combination of the individual antibodies listed in Table 1 below. For example, a lateral flow device may include 628A (available from Bethyl Laboratories, USA; A303-628A) for the capture of GST-Pi and M01 (available from Abcam PLC, Taiwan; H00002950-M01) indirectly conjugated to gold particles using streptavidin/biotin linker system, for the detection of GST-Pi. Testing has demonstrated that this particular combination is able to generate a limit of detection of approximately 20-40 ng/ml with a low background signal.

TABLE 1
Exemplary antibodies that can be used in the assays and methods described herein.

| Supplier | Product Name | Product No. | Abbreviation |
|---|---|---|---|
| Abcam PLC, | GSTP1 purified MaxPab rabbit | H00002950- D01P | “D01P” |
| Taiwan | polyclonal antibody (D01P) | | |
| Abcam PLC, | GSTP1 monoclonal antibody (M03), | H00002950- M03 | “M03” |
| Taiwan | clone S1 | | |
| Abcam PLC, | GSTP1 monoclonal antibody (M01), | H00002950- M01 | “M01” |
| Taiwan | clone 2G6-F6 | | |
| Abcam PLC, | Anti-GST3/GST Pi antibody | ab117885 | “117” |
| Taiwan | | | |
| Abcam PLC, | Anti-GST3/GST Pi antibody | ab150000 | “150” |
| Taiwan | | | |
| Abcam PLC, | Recombinant Anti-GST3/GST Pi | ab245762 | “245” |
| Taiwan | antibody [EPR8263] - BSA and | | |
| | Azide free | | |
| Abcam PLC, | Anti-GST3/GST Pi antibody | ab242014 | “242” |
| Taiwan | [EPR20554] - BSA and Azide free | | |
| Bethyl Labs, USA | GSTP1 Antibody | A303-628A | “628-A” |
| Thermo Fisher | GSTP1 Recombinant Rabbit | MA5-29313 | “313” |
| Scientific, USA | Monoclonal Antibody | | |
| Randox | GSTP1 Recombinant Sheep | MAB10823 | “823” |
| Laboratories, UK | Monoclonal Antibody | | |
| Randox | GSTP1 Recombinant Sheep | MAB10824 | “824” |
| Laboratories, UK | Monoclonal Antibody | | |

In some aspects, lateral flow devices according to the disclosure may be configured such that the capture antibody and/or the detection antibody are present at a concentration of 0.1, 0.2, 0.3, 0.4, 0.5, 0.6, 0.7, 0.8, 0.9, 1.0, 1.1, 1.2, 1.3, 1.4 or 1.5, 1.6, 1.7, 1.8, 1.9 or 2.0 mg/mL (or present at a concentration within a range bounded by any pair of these values) during operation of the device. Additional concentrations may alternatively be used as desired for a given implementation.

In some aspects, the detection antibody may be conjugated to a colored particle (e.g., a gold nanoparticle or red latex particle). Lateral flow devices according to the disclosure may be configured such that conjugated particle is present at an optical density of 1, 2, 3, 4, 5, 6, 7, 8, 9, or 10. For example, the optical density may be 3, 5, or 7.

In some aspects, lateral flow devices according to the disclosure may be configured to measure the concentration of GST-Pi in a biological fluid (e.g., whole blood or serum). The biological fluid may comprise, e.g., a sample of ≤10, 20, 30, 40, or 50 μL of whole blood. In some aspects, the biological fluid may comprise a fraction of whole blood.

In some aspects, lateral flow devices according to the disclosure may be configured to have a lower limit of detection of GST-Pi of 10, 20, 30, 40, 50, 60, 70, 80, 90, or 100 ng/mL. The colorimetric indicator used to detect GST-Pi may be visual to a human or, in some aspects, to an electronic device (e.g., allowing for automated or assisted reading).

In some aspects, lateral flow devices according to the disclosure may comprise a nitrocellulose membrane having a pore size of at least, at most, or about 5, 10, 15, 20, 25, or 30 μm (or having an average pore size within a range bounded by any of these values). Additional pore size may alternatively be used as desired for a given implementation. In some aspects, the capture antibody may be printed onto or otherwise fixed to the nitrocellulose membrane.

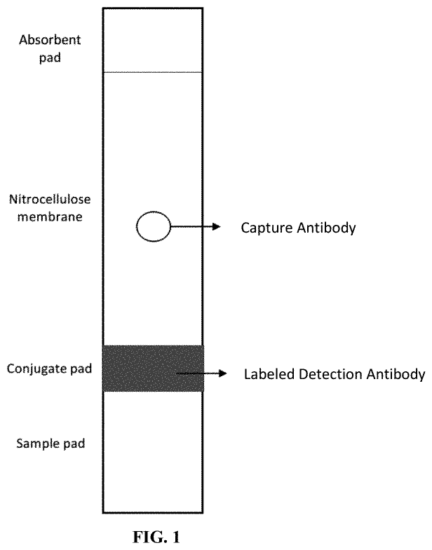

FIG. 1 shows an exemplary lateral flow device according to the disclosure. As illustrated by this figure, a lateral flow device may comprise a sample pad (to receive the sample of biological fluid being tested), a conjugate pad which provides the labeled (detection) antibody, a nitrocellulose membrane which includes the capture antibody (e.g., printed onto the membrane), and an absorbent pad. In some aspects, the absorbent pad is treated with a blocking buffer (e.g., comprising Tris, BSA, and Tween).

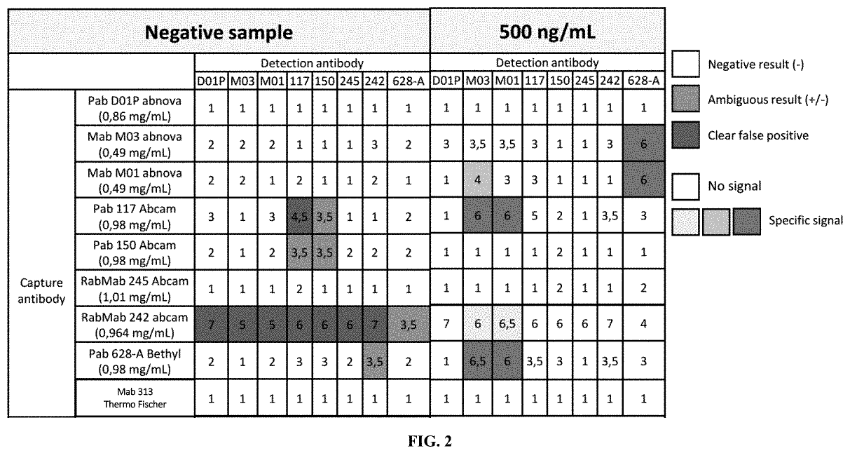

FIG. 2 shows the results of a series of tests which compared various combinations of the antibodies listed in Table 1 as detection and capture antibodies for use in lateral flow devices according to the present disclosure. As illustrated by this table, most combinations of these antibodies failed to produce a viable signal or displayed negative properties (e.g., ambiguous or false positive results). However, a small number of specific pairs of these antibodies produced strong and accurate signals (e.g., the combination of either M01 or M03 as a detection antibody with 628-A as a capture antibody).

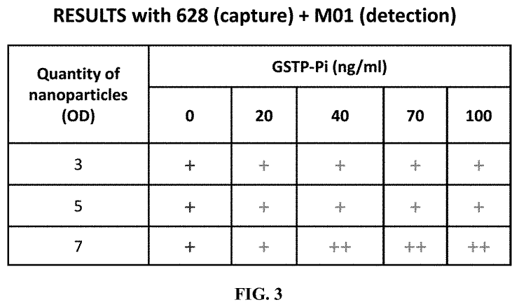

FIG. 3 shows the results of an assay designed to compare the lower limit of detection of GST-Pi using detection antibodies conjugated to nanoparticles at different optical density (OD) levels. As illustrated by these assay, an OD level of 3, 5, or 7 was found to be capable of detecting GST-Pi down to a lower limit of detection of 20 ng/mL, when 628-A was used as a capture antibody with M01 as a detection antibody.

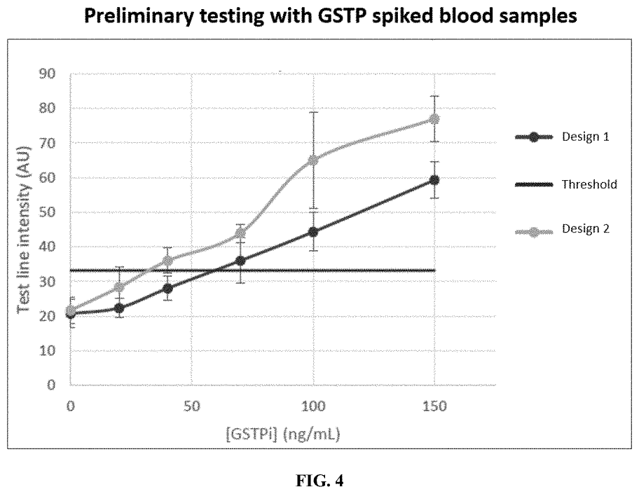

FIG. 4 shows the results of an assay which evaluated the ability of lateral flow devices according to the present disclosure to measure the concentration of GST-Pi in whole blood spiked with GST-Pi at various concentrations. As illustrated by these tests, exemplary devices according to the disclosure are able to produce a linear signal response across the clinically-relevant range of 20-150 ng/ml, rendering them suitable for use in the detection of strokes and for estimating the time of onset.

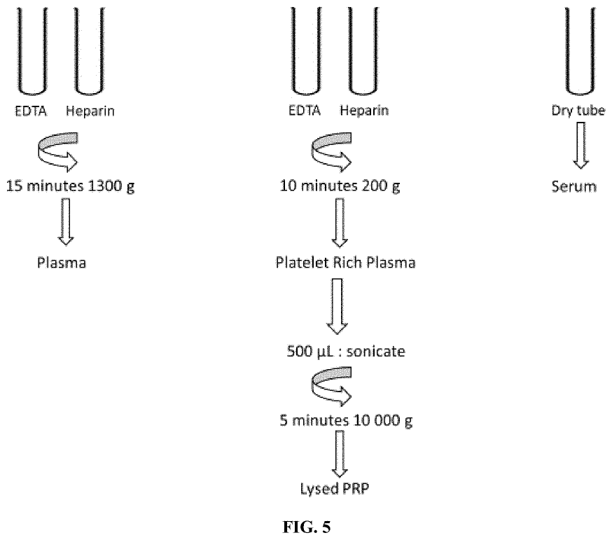

FIG. 5 shows an exemplary protocol for preparing blood fractions to evaluate GST-Pi release from platelets in a biological sample obtained from a human subject that was used to establish the dynamic range of signal expected in severe cases of platelet usage, such as the neutropenia associated with SARS-Cov-2 (COVID-19) infection.

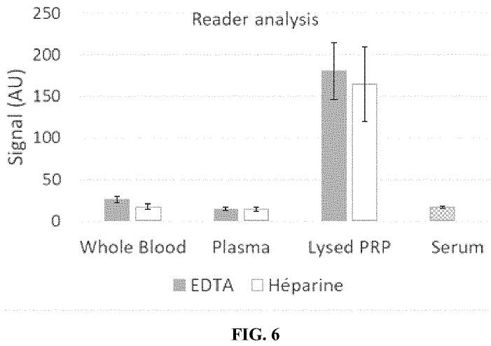

FIG. 6 is a chart showing that levels of GST-Pi measured with a lateral flow device correlated with the platelet levels in blood fractions prepared from a biological sample obtained from a human subject. The release of GST-Pi from platelets during clotting was reflected in a marginally higher signal intensity in serum and whole blood compared with plasma. Highest levels were obtained in a mechanically disrupted, enriched preparation of blood-derived platelets.

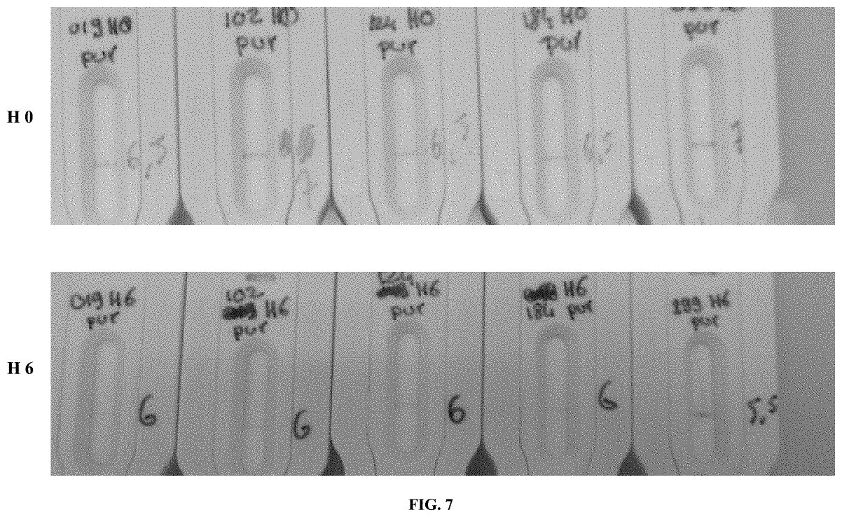

FIG. 7 is a photograph showing the results of a study that evaluated the use of exemplary lateral flow devices according to the present disclosure to measure the concentration of GST-Pi in plasma samples drawn from patients with clinically confirmed stroke at early (<3 hour) and late (>6 hour) time-points. Four individuals showed a slight decrease in GST-Pi levels at the later time-point, whilst one patient demonstrated stable elevation of GST-Pi levels between the two time-points.

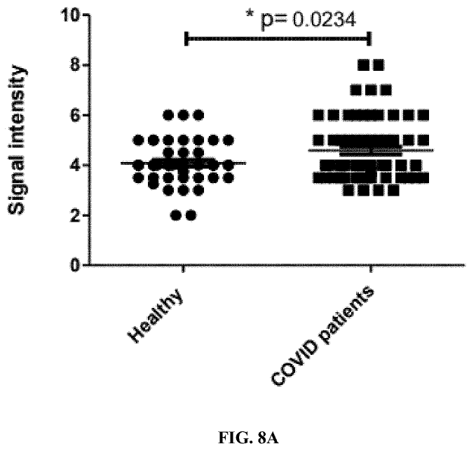

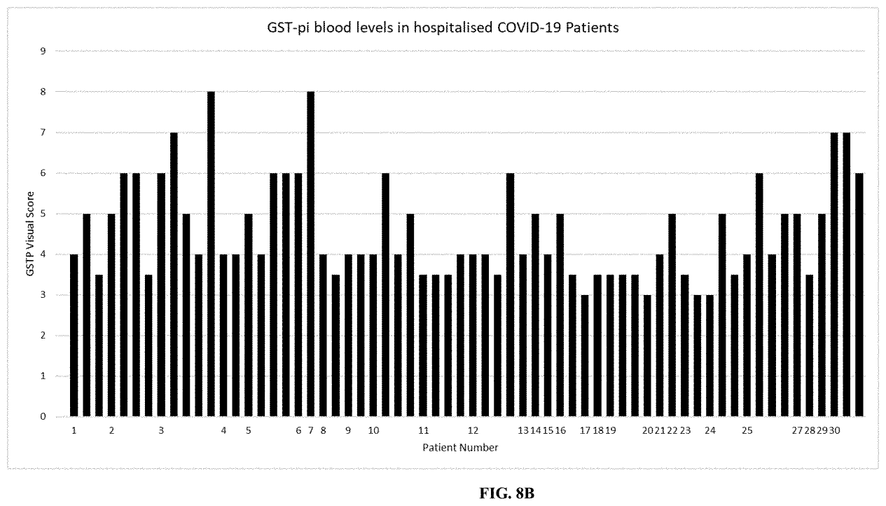

FIG. 8A Is a graph showing the results of a study that evaluated the use of exemplary lateral flow devices according to the present disclosure to measure the concentration of GST-Pi in serum samples drawn from patients with SARS-Cov-2 (COVID-19) infection and healthy controls. The median value in the COVID-19 group is elevated compared with healthy controls, and individual patients demonstrated different temporal evolution of the GST-Pi signal.

FIG. 8B is a graph showing the GST-Pi score for each of 64 serum samples from 30 individual COVID-19 infected patients undergoing treatment in hospital. 15 individual samples showed elevated levels (Score ≥6) and these were drawn from 9 separate patients.

The lateral flow devices described herein may be used to detect whether a subject had had a stroke or transient ischemic attack, and in some aspects may be used to estimate the time of onset such events, allowing for the proper selection and administration of treatment. As such, in some aspects the present devices may be used to determine whether a subject had experienced a stroke within the previous 0.5, 1.0, 2.0, 3.0, 4.0, 5.0, 6.0, 7.0, or 8.0 hours (or within a range bounded by any of these values), and also whether to administer thrombolytic therapy to a subject in need thereof. Additional methods of use will become apparent in view of the totality of the present disclosure.

For example, as noted above the lateral flow devices provided herein may be used for determining the current status, likelihood of progression to severe disease, and monitoring, of a human subject suffering from an ARDS, or to guide the treatment of such subjects. In order to assess the ability of these devices to measure GST-Pi released from platelets, the signal intensity from four different blood fractions—whole blood, plasma, serum and platelet-rich plasma (PRP)—was compared. The effect of EDTA and heparin on the detected levels in all samples except serum was also compared. The protocol and results of this experiment are summarized by FIGS. 5 and 6, respectively.

In short, fresh blood was collected from a healthy male subject into appropriate tubes and processed to generate each blood fraction as shown in FIG. 5. To prepare platelet-enriched plasma, we performed a low-speed centrifugation at 200 G for 10 minutes. The supernatant was removed and sonicated for 5 minutes to lyse platelets and the debris removed by centrifugation at 10,000 G for 5 minutes. The supernatant was then used to measure the level of GST-Pi using a lateral flow device according to the disclosure. In addition, whole blood from a healthy male was collected into standard EDTA or heparin tubes and analyzed directly. To perform the measurements, 20 μl of each blood fraction was loaded onto the sample port of the GST-Pi lateral flow device. Once the blood sample was fully absorbed, the assay running buffer was added and allowed to transport the blood components across the lateral flow membrane. A positive signal develops in the presence of GST-Pi in the blood sample with an intensity of staining proportional to its concentration. All tests were read by eye and with a manual reading device.

As shown by FIG. 6, the levels of GSTP correlated with the level of platelet content, with PRP samples displaying a much stronger signal than the other blood fractions. This experiment also showed that activation of the clotting process in serum and whole blood increased the GST-Pi signal relative to plasma, though there was little difference in levels measured in the presence of the two different anticoagulants.

As evidenced by this study, the lateral flow devices described herein may be used for determining the current status, likelihood of progression to severe disease, and monitoring of treatment effects, in a subject suffering from an ARDS. In some aspects, such methods may comprise: a) obtaining a sample of a biological fluid from a subject suspected of having an ARDS or disseminated vascular coagulation; b) detecting and/or measuring the level of GST-Pi in the sample obtained in step a); and c) determining the current status and/or likelihood of progression to severe disease, of the human subject, based on the level of GST-Pi detected or measured in step b). In some aspects, such methods may be used to monitor the status of the human subject by detecting or measuring the level of GST-Pi in a series of samples of biological fluid obtained from the human subject over a period of time (e.g., collected every 1, 2, 3, 4, 5, 6, 7, 8, 9, 10, 11, 12, 13, 14, 15, 16, 17, 18, 19, 20, 21, 22, 23, 24, 48, or 72 hours). In still further aspects, a determination as to the current status and/or likelihood of progression to severe disease may be made based upon a plurality of samples obtained over the human subject over the aforementioned period of time, or an alternative timespan. The present methods may also be used to determine the effectiveness of a treatment administered to the human subject or to select a treatment method. For example, a treatment method may be selected or changed based upon the GST-Pi level detected and/or measured in a sample of a biological fluid obtained from the subject. Any of the aforementioned methods may advantageously be performed using the lateral flow devices described herein, which allow for the rapid detection and/or measurement of the level of GST-Pi in a sample of biological fluid (e.g., within 5-15 minutes), at the POC, without additional specialized equipment.

### Example 1—Development of GST-Pi Lateral Flow Device (LFD)

Antibodies specific for GST-Pi were purchased from commercial vendors as defined in Table 1. These antibodies were tested in each pairwise combination as both capture and detection antibodies in the LFD. Capture antibodies were printed as discrete lines on a nitrocellulose membrane, which was then cut into appropriate sized strips and assembled into a cassette incorporating the pre-loaded conjugate pad and further absorbent pads for sample loading and buffer wicking according to FIG. 1. The detection antibodies were conjugated to colloidal gold particles and loaded into the conjugate pad during LFD cassette assembly.

For each antibody pair, duplicate LFDs were loaded with 20 μl of buffer alone (Negative sample) or buffer spiked with 500 ng/ml recombinant GST-Pi and left for 15 minutes at room temperature. The color intensity at the capture antibody line was read manually using a reference gold color card (NG Biotech). The results for each pair are shown in FIG. 2, where an intensity >4 is considered positive, and summarized in Table 2 with a ‘+’ indicating a suitable combination for further evaluation.

TABLE 2
Results of pairwise testing of GST-Pi antibodies
for use in a lateral flow device.

| | | Detection Antibody | | | | | | | |
|---|---|---|---|---|---|---|---|---|---|
| | | | | | | | | | 628- |
| | | D01P | M03 | M01 | 117 | 150 | 245 | 242 | A |
| Capture | D01P | − | − | − | − | − | − | − | − |
| Anti- | M03 | − | − | − | − | − | − | − | + |
| body | M01 | − | + | − | − | − | − | − | + |
| | 117 | − | + | + | − | − | − | − | − |
| | 150 | − | − | − | − | − | − | − | − |
| | 245 | − | − | − | − | − | − | − | − |
| | 242 | − | − | − | − | − | − | − | − |
| | 628- | − | + | + | − | − | − | − | − |
| | A | | | | | | | | |

### Example 2—Measurement of Recombinant GST-Pi in Human Plasma

LFDs were manufactured by immobilizing anti-GST-Pi antibody “628-A” onto a nitrocellulose membrane strip in a discrete line within the detection window and absorbing the detection anti-GST-Pi antibody “M03” conjugated to gold particles into a conjugate pad. The nitrocellulose membrane and conjugate pad were then assembled into the cassette with absorbent pads to create the final testing device. Normal human serum was prepared by venepuncture of a healthy adult male collected into standard plastic tubes. Blood was kept at room temperature for 30 minutes and the clot removed by centrifugation and the serum used for preparation of a dilution series of recombinant GST-Pi with final concentrations of 0, 20, 40, 70, 100 and 150 ng/ml. For each concentration of GST-Pi, 20 μl of serum was added to the sample port of three replicate LFDs. Once all serum had been absorbed, two drops of test buffer were added and the LFDs were incubated at room temperature for 15 minutes. The intensity of gold particle staining at the GST-Pi line was measured manually by reference to the gold color card (NG Biotech). Results are shown in FIG. 2 demonstrating the test attained a limit of detection within the required physiological range at 40 ng/ml.

### Example 3—Measurement of GST-Pi in 5 Cases of Ischemic Stroke

To evaluate the performance of a lateral flow device for measurement of GST-Pi for the establishment of the presence and likely time of onset of stroke, we obtained plasma and serum samples from six individuals that were admitted to the hospital with a diagnosis of stroke. All samples had been prepared in accordance with standard clinical practice and were fully consented. For each patient, we also obtained the time from onset of symptoms and National Institute of Health Stroke Score (NIHSS) score (Table 3).

TABLE 3
Sample demographics and GST-Pi scores measured by LFD.

| | | | | Time of onset | Freezing time | | |
|---|---|---|---|---|---|---|---|
| Patient | Age | | Sample | after symptoms | after sampling | GST-Pi Score | GST-Pi Score |
| Code | (yr) | NIHSS | Code | (min) | (min) | (Plasma) | (1/10 Serum) |
| 01-102 | 71 | 20 | H0 | 85 | 255 | 7 | 6.5 |
| 01-102 | | | H6 | 445 | 120 | 6 | 5.5 |
| 01-184 | 85 | 18 | H0 | 80 | 150 | 6.5 | 6 |
| 01-184 | | | H6 | 440 | 95 | 6 | 5 |
| 01-299 | 63 | 17 | H0 | 95 | 155 | 7 | 6.5 |
| 01-299 | | | H6 | 455 | 115 | 5.5 | 6 |
| 01-108 | 79 | 17 | H0 | 100 | 175 | ND | 6 |
| 01-108 | | | H6 | 460 | | ND | 4 |
| 01-019 | 84 | 11 | H0 | 85 | 160 | 6.5 | ND |
| 01-019 | | | H6 | 85 | 100 | 6 | ND |
| 01-124 | 78 | 9 | H0 | 60 | 120 | 6.5 | 6 |
| 01-124 | | | H6 | 420 | 120 | 6 | 6 |

LFDs were manufactured by immobilizing anti-GST-Pi antibody “823” onto a nitrocellulose membrane strip in a discrete line within the detection window and absorbing the detection anti-GST-Pi antibody “M01” conjugated to gold particles into a conjugate pad. The nitrocellulose membrane and conjugate pad were then assembled into the cassette with absorbent pads to create the final testing device. To measure GST-Pi levels, a volume of 20 μl of plasma or pre-diluted serum was added to the sample port of each of three replicate LFD cassettes for each sample and allowed to absorb into the sample pad. Once each sample was fully absorbed, two drops of test buffer were added and LFDs were incubated at room temperature for 15 minutes. The intensity of gold particle staining at the GST-Pi line was measured manually by reference to the gold color card (NG Biotech). Results are shown in FIG. 7 and Table 3. As expected, levels of GST-Pi decreased between the early and late samples for all five plasma samples. Similarly, in the pre-diluted serum, a reduction in GST-Pi levels was observed for four individuals whilst the fifth showed a stable, elevated level.

### Example 4—Measurement of GST-Pi in COVID-19 Infected Patient Samples

To explore the level of GST-Pi in the serum of COVID-19 patients, we obtained a cohort of 64 samples from 30 infected individuals admitted to the hospital (Table 4) along with a single sample from each of 44 healthy donors.

TABLE 4
Details of 64 serum samples from 30 COVID-19
infected patients used for GST-Pi analysis.

| Patient | Sample | Day after | GST- | COVID-19 Antibody Score | |
|---|---|---|---|---|---|
| ID | No | symptoms onset | Pi | IgM | IgG |
| 1 | 1 | 6 | 4 | 1 | 1 |
| 1 | 2 | 10 | 5 | 7 | 6 |
| 1 | 3 | 17 | 3.5 | 8 | 10 |
| 2 | 1 | 14 | 5 | 8 | 10 |
| 2 | 2 | 24 | 6 | 7 | 10 |
| 2 | 3 | 27 | 6 | 7 | 10 |
| 2 | 4 | 30 | 3.5 | 7 | 10 |
| 3 | 1 | 13 | 6 | 7 | 9 |
| 3 | 2 | 29 | 7 | 6 | 10 |
| 3 | 3 | 31 | 5 | 5 | 10 |
| 3 | 4 | 34 | 4 | 4 | 9 |
| 3 | 5 | 37 | 8 | 5 | 10 |
| 4 | 1 | 9 | 4 | 5 | 10 |
| 4 | 3 | 15 | 4 | 6 | 10 |
| 5 | 1 | 6 | 5 | 7 | 4.5 |
| 5 | 2 | 18 | 4 | 9 | 10 |
| 5 | 3 | 35 | 6 | 9 | 10 |
| 5 | 4 | 42 | 6 | 7 | 10 |
| 6 | 1 | 16 | 6 | 7 | 10 |
| 7 | 1 | 21 | 8 | 8 | 10 |
| 8 | 1 | 4 | 4 | 7 | 10 |
| 8 | 2 | 11 | 3.5 | 8 | 10 |
| 9 | 3 | 19 | 4 | 6 | 10 |
| 9 | 4 | 28 | 4 | 6 | 10 |
| 10 | 1 | 4 | 4 | 9 | 10 |
| 10 | 2 | 15 | 6 | 8 | 10 |
| 10 | 3 | 18 | 4 | 8 | 10 |
| 10 | 4 | 21 | 5 | 7 | 10 |
| 11 | 1 | 9 | 3.5 | 8 | 10 |
| 11 | 2 | 16 | 3.5 | 8 | 10 |
| 11 | 3 | 21 | 3.5 | 8 | 10 |
| 11 | 4 | 25 | 4 | 7 | 10 |
| 12 | 1 | 1 | 4 | 1 | 1 |
| 12 | 2 | 3 | 4 | 1 | 1 |
| 12 | 3 | 6 | 3.5 | 1 | 1 |
| 12 | 4 | 10 | 6 | 4.5 | 10 |
| 13 | 2 | 23 | 4 | 6 | 10 |
| 14 | 1 | 15 | 5 | 6 | 10 |
| 15 | 1 | 10 | 4 | 6 | 6 |
| 16 | 1 | 42 | 5 | 4 | 10 |
| 16 | 2 | 52 | 3.5 | 3 | 10 |
| 17 | 2 | 16 | 3 | 7 | 10 |
| 18 | 1 | 22 | 3.5 | 7 | 10 |
| 19 | 1 | 42 | 3.5 | 5.5 | 10 |
| 19 | 2 | 45 | 3.5 | 6 | 10 |
| 19 | 3 | 52 | 3.5 | 6 | 10 |
| 20 | 2 | 26 | 3 | 8 | 10 |
| 21 | 3 | 34 | 4 | 4 | 10 |
| 22 | 1 | 23 | 5 | 6 | 10 |
| 23 | 1 | 9 | 3.5 | 3 | 6.5 |
| 23 | 2 | 18 | 3 | 6 | 10 |
| 24 | 1 | 5 | 3 | 4.5 | 9 |
| 24 | 2 | 9 | 5 | 4 | 7 |
| 24 | 3 | 12 | 3.5 | 4.5 | 9 |
| 25 | 1 | 3 | 4 | 7 | 10 |
| 25 | 2 | 6 | 6 | 7 | 10 |
| 25 | 3 | 16 | 4 | 7 | 10 |
| 25 | 4 | 18 | 5 | 6.5 | 10 |
| 27 | 1 | 24 | 5 | 4.5 | 10 |
| 28 | 1 | 24 | 3.5 | 6 | 10 |
| 29 | 1 | 7 | 5 | 2 | 1 |
| 30 | 1 | 2 | 7 | 4.5 | 1 |
| 30 | 3 | 13 | 7 | 9 | 10 |
| 30 | 4 | 16 | 6 | 9 | 10 |

LFD cassettes were manufactured using “823” as the capture antibody and M01 conjugated to colloidal gold for detection. For each sample, a 10 μl aliquot of serum was added to the sample port. Once the sample had been fully absorbed, two drops of test buffer were added to the sample port and devices incubated for 10 minutes at room temperature. The intensity of staining was read manually by reference to the gold color card (NG Biotech). Results are shown in FIG. 8. When data of the two populations were analyzed, (FIG. 8A) there was a clear separation of the median GST-Pi serum levels with an elevation in the COVID-19 population. Using a cut-off score of 6 there were 15/64 positive samples from COVID-19 patients and 3/44 from the control group. Within the COVID-19 infected cohort, the elevation of GST-Pi was seen in 9 of 30 individual patients. In these individuals the elevation of GST-Pi did not show any association with time from onset, length of hospitalization or anti-COVID-19 antibody titre (determined by experimental lateral flow test).

In the interest of clarity, not all of the routine features of the exemplary aspects are disclosed herein. It will be appreciated that in the development of any actual implementation of the present disclosure, numerous implementation-specific decisions must be made in order to achieve specific goals, which will vary for different implementations. It will be appreciated that such efforts might be complex and time-consuming, but would nevertheless be a routine undertaking for those of ordinary skill in the art having the benefit of this disclosure.

Furthermore, it is to be understood that the phraseology or terminology used herein is for the purpose of description and not of restriction, such that the terminology or phraseology of the present specification is to be interpreted in light of the teachings and guidance presented herein, in combination with the knowledge available to a person of ordinary skill in the relevant art(s) at the time of invention. Moreover, it is not intended for any term in the specification or claims to be ascribed an uncommon or special meaning, unless explicitly set forth as such in the specification.

The various aspects disclosed herein encompass present and future known equivalents to the known structural and functional elements referred to herein by way of illustration. Moreover, while aspects and applications have been shown and described, it would be apparent to those skilled in the art having the benefit of this disclosure that many more modifications than those mentioned above are possible without departing from the inventive concepts disclosed herein. For example, one of ordinary skill in the art would readily appreciate that individual features from any of the exemplary aspects disclosed herein may be combined to generate additional aspects that are in accordance with the inventive concepts disclosed herein.

A transitional terms “comprising,” “consisting essentially of,” and “consisting of,” when used in the appended claims, in original and amended form, define the claim scope with respect to what unrecited additional claim elements or steps, if any, are excluded from the scope of the claims. The term “comprising” is intended to be inclusive or open-ended and does not exclude any additional, unrecited element, method, step or material. The term “consisting of” excludes any element, step or material other than those specified in the claim and, in the latter instance, impurities ordinarily associated with the specified material(s). The term “consisting essentially of” limits the scope of a claim to the specified elements, steps or material(s) and those that do not materially affect the basic and novel characteristic(s) of the claimed invention. All compositions, devices, methods, and kits described herein that embody aspects of the present invention can, in alternate embodiments, be more specifically defined by any of the transitional terms “comprising,” “consisting essentially of,” and “consisting of” As such, any reference to the transitional term “comprising” in the present disclosure is understood as also contemplating alternative aspects which “consist of,” or “consist essentially of,” the same recited elements.

Although illustrative exemplary aspects have been shown and described, a wide range of modification, change and substitution is contemplated in the foregoing disclosure and in some instances, some features of the embodiments may be employed without a corresponding use of other features. Accordingly, it is appropriate that the appended claims be construed broadly and in a manner consistent with the scope of the embodiments disclosed herein.

1. A lateral flow immunoassay device for detecting and/or measuring the concentration of glutathione S-transferase Pi (GST-Pi) in a sample of a biological fluid, said device comprising a test strip for detecting GST-Pi in the sample, wherein the test strip comprises:
a) a sample pad, wherein the sample pad comprises an absorbent material and is configured to receive the sample;
b) a conjugate pad configured to store a detection antibody specific for GST-Pi and to release at least a portion of the stored detection antibody in the presence of a liquid, wherein the detection antibody is conjugated to a colored label; and
c) a colorimetric indicator site positioned downstream from the absorbent pad, wherein said colorimetric indicator site comprises a capture antibody specific for GST-Pi fixed to the test strip.

2. The lateral flow immunoassay device of claim 1, wherein the device further comprises a lancet with a capillary channel.

3. The lateral flow immunoassay device of claim 1, wherein at least a portion of the test strip comprises a nitrocellulose membrane.

4. The lateral flow immunoassay device of claim 1, wherein at least a portion of the test strip comprises a nitrocellulose membrane with an average pore size of at least, at most, or about 5, 10, 15, 20, 25, or 30 μm.

5. The lateral flow immunoassay device of claim 1, wherein the capture antibody and the detection antibody are configured to bind to different moieties of GST-Pi.

6. The lateral flow immunoassay device of claim 1, wherein the capture antibody and/or the detection antibody are each selected from any of the antibodies described herein.

7. The lateral flow immunoassay device of claim 1, wherein the colored label comprises a gold or latex nanoparticle.

8. The lateral flow immunoassay device of claim 1, wherein the colored label is present at an optical density of 1, 2, 3, 4, 5, 6, 7, 8, 9, or 10 OD.

9. The lateral flow immunoassay device of claim 1, wherein the capture antibody and/or the detection antibody are present at a concentration of 0.1, 0.2, 0.3, 0.4, 0.5, 0.6, 0.7, 0.8, 0.9, 1.0, 1.1, 1.2, 1.3, 1.4 or 1.5, 1.6, 1.7, 1.8, 1.9 or 2.0 mg/mL.

10. The lateral flow immunoassay device of claim 1, wherein the capture antibody comprises A303-628A and the detection antibody comprises H00002950-M01.

11. The lateral flow immunoassay device of claim 1, wherein the capture antibody comprises MAB10823 and the detection antibody comprises H00002950-M01.

12. The lateral flow immunoassay device of claim 1, wherein the biological fluid comprises a sample of whole blood from a human subject.

13. The lateral flow immunoassay device of claim 1, wherein the GST-Pi comprises a polypeptide sequence of SEQ ID NO: 1.

14. The lateral flow immunoassay device of claim 1, wherein the GST-Pi comprises a polypeptide sequence sharing at least 90%, 95%, or 99% sequence identity with SEQ ID NO: 1.

15. A method for determining whether a subject has had a stroke or ischemic attack, comprising:
a) obtaining a sample of a biological fluid from a subject suspected of having had a stroke or ischemic attack;
b) applying at least a portion of the sample to any lateral flow device described herein; and
c) detecting a level and/or concentration of GST-Pi in the sample using the lateral flow device.

16. The method of claim 15, further comprising:
d) determining an estimated time of onset of the stroke or ischemic attack based on the level and/or concentration of GST-Pi detected in step c).

17. The method of claim 15, further comprising:
d) repeating steps a) through c) at a subsequent time-point; and
e) determining whether the level and/or concentration of GST-Pi detected in the sample has increased or decreased over time.

18. The method of claim 15, further comprising:
d) determining an estimated time of onset of the stroke or ischemic attack based on the level and/or concentration of GST-Pi detected in step c); and
e) selecting a treatment for the subject based on the estimated time of onset determined in step d).

19. The method of claim 18, wherein the treatment comprises administration of a thrombolytic therapy.

20. A method for determining the current status and/or likelihood of progression to severe disease, of a human subject suffering from an acute respiratory distress syndrome (ARDS), comprising:
a) obtaining at least one sample of a biological fluid from a subject suspected of having an ARDS and/or disseminated vascular coagulation;
b) detecting and/or measuring the level of GST-Pi in the at least one sample obtained in step a); and
c) determining the current status and/or likelihood of progression to severe disease, of the human subject, based on the level of GST-Pi detected or measured in step b).

21. The method of claim 20, wherein the ARDS is COVID-19.

22. The method of claim 20, wherein step a) comprises obtaining a plurality of samples from the human subject at different time-points.

23. The method of claim 21, wherein step a) comprises obtaining a plurality of samples from the human subject at different time-points.

24. The method of claim 22, wherein the plurality of samples comprises samples obtained from the human subject:
a) every 1, 2, 3, 4, 5, 6, 7, 8, 9, 10, 11, 12, 13, 14, 15, 16, 17, 18, 19, 20, 21, 22, 23, 24, 48, or 72 hours;
b) at least once per day, over a plurality of days; or
c) before or after a treatment for the ARDS and/or disseminated vascular coagulation is administered to the human subject.

25. A method for determining whether a subject has had a head injury or concussion, comprising:
a) obtaining a sample of a biological fluid from a subject suspected of having had a head injury or concussion;
b) applying at least a portion of the sample to any lateral flow device described herein; and
c) detecting a level and/or concentration of GST-Pi in the sample using the lateral flow device.

26. The method of claim 25, further comprising:
d) determining an estimated time of occurrence of the head injury or concussion based on the level and/or concentration of GST-Pi detected in step c).

27. The method of claim 25, further comprising:
d) repeating steps a) through c) at a subsequent time-point; and
e) determining whether the level and/or concentration of GST-Pi detected in the sample has increased or decreased over time.

28. The method of claim 25, further comprising:
d) determining an estimated time of occurrence of the head injury or concussion based on the level and/or concentration of GST-Pi detected in step c); and
e) selecting a treatment for the subject based on the estimated time of onset determined in step d).

29. The method of claim 28, wherein the treatment comprises administration of a thrombolytic therapy.

30. The method of claim 25, wherein step a) comprises obtaining a plurality of samples from the human subject at different time-points.

31. The method of claim 28, wherein step a) comprises obtaining a plurality of samples from the human subject at different time-points.

32. The method of claim 30, wherein the plurality of samples comprises samples obtained from the human subject:
a) every 1, 2, 3, 4, 5, 6, 7, 8, 9, 10, 11, 12, 13, 14, 15, 16, 17, 18, 19, 20, 21, 22, 23, 24, 48, or 72 hours;
b) at least once per day, over a plurality of days; or
c) before or after a treatment for the ARDS and/or disseminated vascular coagulation is administered to the human subject.

33. A method for monitoring the effect of treating a human subject suffering from an ARDS, comprising:
a) obtaining a sample of a biological fluid from a subject suspected of having an ARDS, before and/or after a treatment for the ARDS is administered to the subject;
b) detecting or measuring the level of GST-Pi in the sample obtained in step a); and
c) determining whether the treatment is likely to slow and/or reduce the likelihood of progression to severe disease, based on the level of GST-Pi detected or measured in step b).

34. The method of claim 33, wherein step a) is performed before or after a treatment for the ARDS is administered to the human subject.

35. The method of claim 33, wherein the ARDS is COVID-19.

36. The method of claim 34, wherein the ARDS is COVID-19.

37. A kit comprising for detecting and/or measuring the concentration of glutathione S-transferase Pi (GST-Pi) in a sample of a biological fluid, comprising:
a) a lateral flow immunoassay device comprising a test strip for detecting GST-Pi in the sample, wherein the test strip comprises:
i) a sample pad, wherein the sample pad comprises an absorbent material and is configured to receive the sample;
ii) a conjugate pad configured to store a detection antibody specific for GST-Pi and to release at least a portion of the stored detection antibody in the presence of a liquid, wherein the detection antibody is conjugated to a colored label; and
iii) a colorimetric indicator site positioned downstream from the absorbent pad, wherein said colorimetric indicator site comprises a capture antibody specific for GST-Pi fixed to the test strip; and
b) one or more buffers.

38. The kit of claim 37, wherein the GST-Pi comprises the polypeptide sequence of SEQ ID NO: 1.

39. The kit of claim 37, wherein the GST-Pi comprises a polypeptide sequence sharing at least 90%, 95%, or 99% sequence identity with SEQ ID NO: 1.
